(19) United States
(12) Patent Application Publication
(10) Pub. No.: US 2026/0283893 A1
(43) Pub. Date: Sep. 24, 2026

(54) MASSAGE DEVICE WITH SWING ARMS OF PORTABLE SLEEP PILLOW

(71) Applicant: XIAMEN VANFLY HEALTH SCIENCE & TECHNOLOGY CO., LTD, XIAMEN (CN)

(72) Inventors: YING CHEN, XIAMEN, FUJIAN (CN); QIXIAN LIN, XIAMEN, FUJIAN (CN)

(21) Appl. No.: 19/189,188

(22) Filed: Apr. 24, 2025

(30) Foreign Application Priority Data
Mar. 20, 2025 (CN) 202510333058.6

(51) Int. Cl. A61H 7/00; A47G 9/10; A61N 5/06

(52) U.S. Cl. CPC A61H 7/007; A47G 9/1045; A47G 9/1081; A61N 5/0625; A61H 2007/009; A61H 2201/0134; A61H 2201/0157; A61H 2201/0192; A61H 2201/0207; A61H 2201/10; A61H 2201/1215; A61H 2201/14; A61H 2201/1609; A61H 2201/1614; A61H 2201/1623; A61H 2201/1676; A61H 2201/5058; A61H 2205/04; A61H 2205/062; A61H 2205/081; A61N 2005/0654

(57) ABSTRACT

A massage device with swing arms of a portable sleep pillow relates to a technical field of physical therapy devices and includes a base, two neck massage heads, two shoulder massage heads, two trapezius massage heads, and a transmission portion. The neck massage heads are obliquely disposed, and an included angle between the two neck massage heads faces upward. The shoulder massage heads and the trapezius massage heads are horizontally disposed. The shoulder massage heads are disposed outside the neck massage heads. The trapezius massage heads are respectively disposed below the shoulder massage heads. The transmission portion is disposed on the base. Reversing pieces are disposed inside the transmission portion. The massage device provides multi-angle massage for the user. When the massage device is not in a massage state, the shoulder massage heads and the two trapezius massage heads do not contact the neck of the user.

### TECHNICAL FIELD

The present disclosure relates to a technical field of physical therapy devices, in particular to a massage device with swing arms of a portable sleep pillow.

### BACKGROUND

In modern life, due to long hours of desk work, use of electronic devices, and poor sleeping postures, more and more people have health problems such as neck and shoulder fatigue, muscle stiffness, and even cervical spondylosis. In order to relieve shoulders and neck discomfort, people often use massage pillows to massage their shoulders and necks.

Conventional massage pillows mostly perform massage through rotation, rolling, or vibration. A massage method thereof is simple and fixed, which is not easy to meet user needs for massage intensity and method. In addition, when in a non-working state, massage heads of the conventional massage pillows are protruding, which cause additional pressure on the necks of the users. When the user sleeps on a conventional massage pillow, the massage heads protruding outward press against a head or a neck of the user, causing discomfort to the user. Moreover, the conventional massage pillows are generally not detachable. After long-term use, sweat stains and dust may accumulate in dead corners of the conventional massage pillows. Since the conventional massage pillows are not allowed to be washed, service life of the conventional massage pillows is limited.

### SUMMARY

The present disclosure provides a massage device with swing arms of a portable sleep pillow. Through multi-angle massage and hideable configurations of massage heads, a user is allowed to adjust the portable pillow between a massage state and a sleep state freely.

To solve above problems, the present disclosure provides a massage device with swing arms of a portable sleep pillow. The massage device comprises a base, two neck massage heads, two shoulder massage heads, two trapezius massage heads, and a transmission portion.

The two neck massage heads are obliquely disposed, and an included angle between the two neck massage heads faces upward. The two shoulder massage heads and the two trapezius massage heads are horizontally disposed. The two shoulder massage heads are disposed outside the two neck massage heads. The two trapezius massage heads are respectively disposed below the two shoulder massage heads.

The transmission portion is disposed on the base and configured to drive the two neck massage heads, the two shoulder massage heads, and the two trapezius massage heads to perform kneading actions. Reversing pieces are disposed inside the transmission portion. The reversing pieces are configured to adjust the kneading actions of the two neck massage heads and the two shoulder massage heads to increase diversity of the kneading actions.

When the kneading actions are not performed, the two neck massage heads are configured to support a neck of a user, and the two shoulder massage heads and the two trapezius massage heads do not contact the neck and shoulders of the user, so as to reduce discomfort of the user.

Furthermore, a protective shell is fixedly connected to an upper surface of the base. Two first swing arms are disposed on the protective shell. The two neck massage heads are respectively fixedly connected to surfaces of the two first swing arms. The two shoulder massage heads are respectively disposed on end portions of the two first swing arms. Universal ball racks are respectively fixedly disposed on bottom portions of the two first swing arms. Ball grooves are defined in a surface of the protective shell. Limiting linear grooves are symmetrically defined in an inner wall surface of each of the ball grooves. Limiting protruding blocks are symmetrically and fixedly connected to a surface of each of the universal ball racks. A spherical part of each of the universal ball racks is disposed in a corresponding one of the ball grooves. The limiting protruding blocks are respectively disposed in the limiting linear grooves. The reversing pieces are disposed below the two universal ball racks.

Furthermore, a battery compartment is mounted on the surface of the protective shell. A brushless motor is mounted inside the protective shell. A spiral gear rod is fixedly connected to an end portion of an output shaft of the brushless motor, first rotating shafts are symmetrically disposed inside the protective shell. The first rotating shafts are rotatably connected to the protective shell through circular grooves symmetrically defined in an inner wall of the protective shell.

The massage device further comprises two helical gears and two first gears. A surface of each of the first rotating shafts is fixedly connected to a corresponding one of the helical gears and a corresponding one of the first gears from top to bottom. The two helical gears are engaged with the spiral gear rod. Two second gears are symmetrically disposed inside the protective shell. Each of the second gears is engaged with a corresponding one of the first gears, and the reversing pieces are respectively disposed on surfaces of the two second gears.

Furthermore, each of the second gears is rotatably disposed in a corresponding one of the circular grooves through a protruding ring disposed on a bottom surface thereof.

The reversing pieces comprise a first reversing block and a second reversing block. The first reversing block and the second reversing block are respectively disposed on the surfaces of the two second gears. First cylinders are respectively protruded from an inclined surface of the first reversing block and an inclined surface of the second reversing block. Each of the first cylinders is inserted into a first columnar groove defined in a bottom surface of a corresponding one of the universal ball racks.

Furthermore, the first reversing block is inserted into a second columnar groove defined in an upper surface of a first one of the two second gears through a second cylinder protruding from a bottom surface of the first reversing block. The second reversing block is fixedly connected to an upper surface of a second one of the two second gears. An adjusting groove is defined in the first reversing block, the adjusting groove is in an incomplete ring shape, and an adjusting block is disposed in the adjusting groove. The adjusting block is fixedly connected to the first one of the two second gears.

Furthermore, the massage device further comprises eccentric blocks, eccentric brackets, and second swing arms. Each of the eccentric blocks is fixedly connected in the protruding ring on the bottom surface of a corresponding one of the second gears. Each of the eccentric brackets is rotatably connected to a shaft disposed on a bottom surface of a corresponding one of the eccentric blocks through a first bearing. Hinge seats are symmetrically and fixedly connected to the surface of the protective shell.

A U-shaped portion is disposed on a surface, away from the protective shell, of each of the hinge seats, and each of the second swing arms is hinged into a corresponding U-shaped portion.

Sliding grooves are defined in surfaces of the hinge seats. Sliding blocks are respectively slidable in the sliding grooves. A bottom end of each of the sliding blocks is fixedly connected to the surface of a corresponding one of the eccentric brackets. Guide ball head rods are respectively connected to surfaces of the second swing arms. Guide grooves are respectively defined in the eccentric brackets, a ball head of each of the guide ball head rods is slidable in a corresponding one of the guide grooves. Each of the trapezius massage heads is disposed on one end of a corresponding one of the second swing arms.

Furthermore, a Hall sensor is mounted on the surface of the protective shell. An induction magnetic column is embedded in an upper surface of one of the second gears closer to the Hall sensor. A printed circuit board (PCB) control box is fixedly connected to the surface of the protective shell. A controller is disposed on one side of the protective shell, and a heating lamp board is disposed above the protective shell.

Furthermore, cloth sleeves are respectively disposed above the ball grooves. The cloth sleeves are elastic to be elastically deformed under the kneading actions of the two first swing arms, each of the cloth sleeves is configured to accommodate a corresponding one of the first swing arms, a corresponding one of the neck massage heads, and a corresponding one of the shoulder massage heads. Massage protrusions are evenly and fixedly connected to outer surfaces of the cloth sleeves. Sizes of the massage protrusions gradually increase along a circumference of each of the cloth sleeves.

Furthermore, limiting buckles are symmetrically and fixedly disposed on two sides of each of the ball grooves defined on the surface of the protective shell. Elastic supporting strips are disposed above the ball grooves, each of the elastic supporting strips is slidably connected to corresponding limiting buckles through T-shaped limiting grooves respectively defined in surfaces of the corresponding limiting buckles. Each of the cloth sleeves is fixedly connected to an upper surface of a corresponding one of the elastic supporting strips.

When the two first swing arms drive the two neck massage heads to perform the kneading actions, the massage protrusions massage the neck. By adjusting positions of the elastic supporting strips relative to the limiting buckles, the massage protrusions do not contact the neck of the user or at least partially contact the neck of the user.

Furthermore, fixed cloth strips are evenly and fixedly connected to an inner wall of each of the cloth sleeves. The fixed cloth strips of each of the cloth sleeves are disposed on one end of each of the cloth sleeves away from the corresponding one of the elastic supporting strips. The fixed cloth strips of each of the cloth sleeves are configured to fix the one end of each of the cloth sleeves to one side of the massage device away from the protective shell.

In the massage device of the present disclosure, two neck massage heads are able to knead and massage the neck of the user, and in the non-massage state, the two neck massage heads are configured to support the neck. Further, the two trapezius massage heads and the two shoulder massage heads are disposed horizontally, so that the two trapezius massage heads and the two shoulder massage heads extend toward the neck. In the non-massage state or a power-off state, positions of the two trapezius massage heads and the two shoulder massage heads are adjusted, so that the two trapezius massage heads and the two shoulder massage heads do not contact the neck of the user, thereby avoiding discomfort caused by the two trapezius massage heads and the two shoulder massage heads when the user rests his/her head on the massage device for sleeping. As a result, sleeping comfort of the user is improved. By configuring the reversing pieces, the two first swing arms are allowed to swing synchronously or asynchronously, which increases overall massage techniques of the two neck massage heads, the two shoulder massage heads and the two trapezius massage heads. By providing the cloth sleeves and the massage protrusions, dust and sweat stains are prevented from accumulating on the massage device, and hygiene of the massage device is maintained.

### BRIEF DESCRIPTION OF DRAWINGS

FIG. 1 is a schematic diagram of a portable sleep pillow according to one embodiment of the present disclosure.

FIG. 2 is an exploded schematic diagram of the portable sleep pillow according to one embodiment of the present disclosure.

FIG. 3 is an exploded schematic diagram of a massage device with swing arms of the portable sleep pillow according to one embodiment of the present disclosure.

FIG. 4 is a schematic diagram of a universal ball rack mounted on a protective shell according to one embodiment of the present disclosure.

FIG. 5 is a partial exploded schematic diagram of a universal ball rack mounted on a protective shell according to one embodiment of the present disclosure.

FIG. 6 is a schematic diagram of a first reversing block and a second gear according to one embodiment of the present disclosure.

FIG. 7 is a partial exploded schematic diagram of a massage device with swing arms of the portable sleep pillow according to one embodiment of the present disclosure.

FIG. 8 is a schematic diagram of limiting buckles mounted on the protective shell according to one embodiment of the present disclosure.

FIG. 9 is a schematic diagram of fixed cloth strips according to one embodiment of the present disclosure.

FIG. 10 is a cross-sectional schematic diagram of the massage device according to one embodiment of the present disclosure, where trapezius massage heads thereof are in a horizontal state.

FIG. 11 is a cross-sectional schematic diagram of the massage device according to one embodiment of the present disclosure, where the trapezius massage heads thereof are in an inclined state.

### IN THE DRAWINGS

101—base; 102—neck massage head; 103—shoulder massage head; 104—trapezius massage head; 201—protective shell; 202—battery compartment; 203—brushless motor; 204—spiral gear rod; 205—first rotating shaft; 206—helical gear; 207—first gear; 208—second gear; 209—universal ball rack; 210—first swing arm; 211—ball groove; 212—limiting linear groove; 213—limiting protruding blocks; 214—circular groove; 301—reversing piece; 3011—first reversing block; 3012—second reversing block; 3013—adjusting groove; 3014—adjusting block; 401—eccentric block; 402—eccentric bracket; 403—second swing arm; 405—hinge seat; 406—sliding groove; 408—sliding block; 409—guide groove; 410—guide ball head rod; 501—Hall sensor; 502—induction magnetic column; 503—PCB control box; 504—controller; 505—heating lamp board; 601—limiting buckle; 602—elastic supporting strip; 603—cloth sleeve; 604—massage protrusion; 605—fixed cloth strip; 801—sponge pillow body.

### DETAILED DESCRIPTION

Exemplary embodiments of the present disclosure are described in details below with reference to accompanying drawings. Although the exemplary embodiments of the present disclosure are shown in the accompanying drawings, it should be understood that the present disclosure can be implemented in various forms and should not be limited by the embodiments set forth herein. On the contrary, these embodiments are provided to enable a more thorough understanding of the present disclosure and to fully convey the scope of the present disclosure to those skilled in the art.

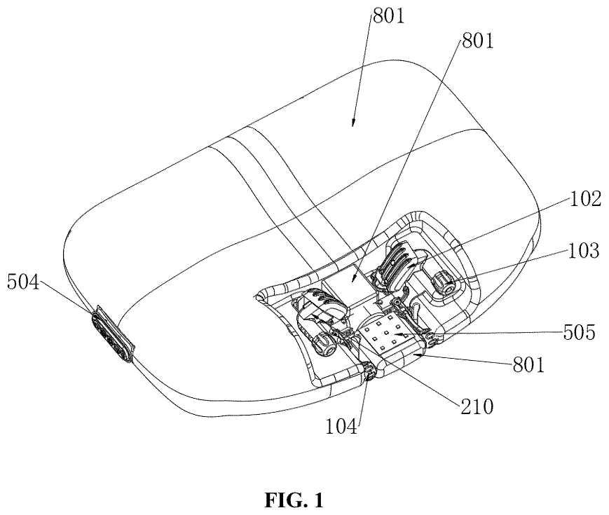

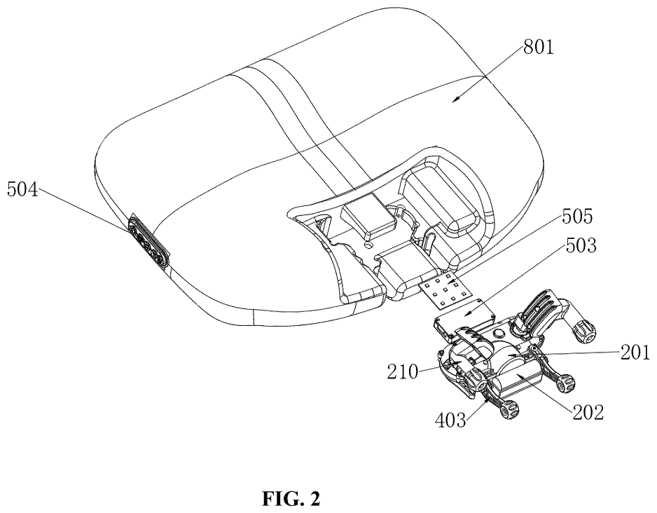

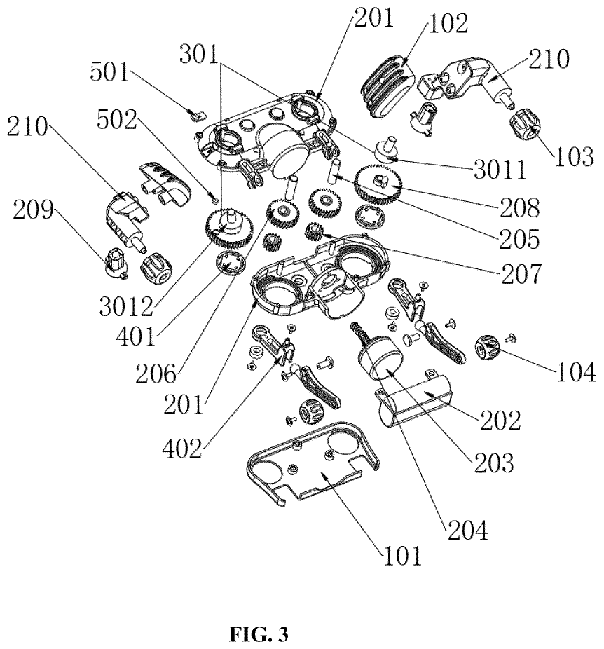

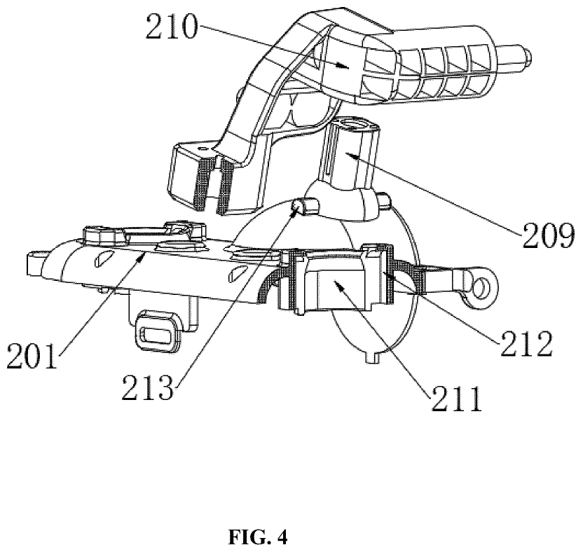

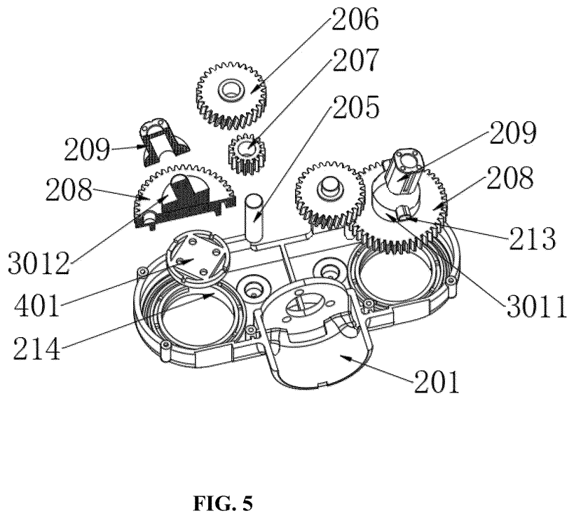

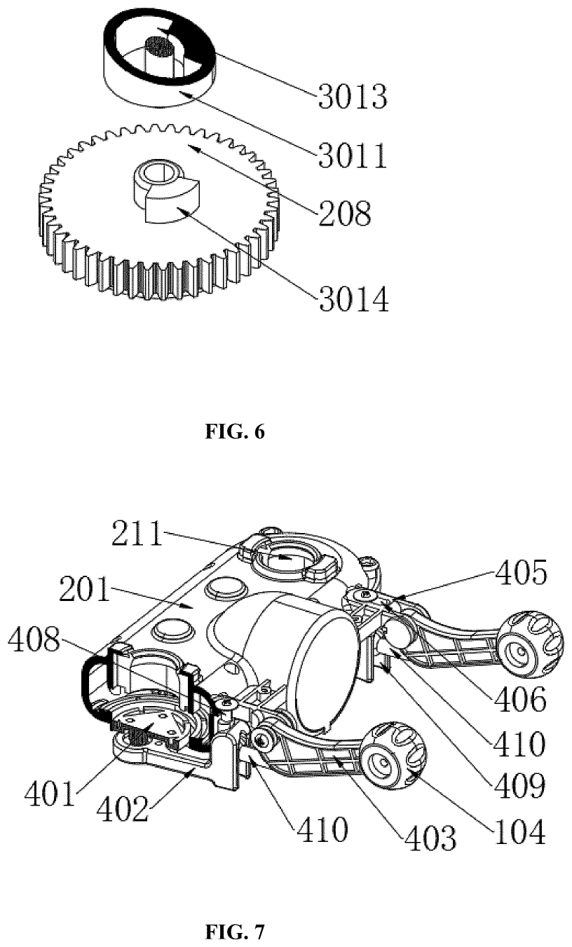

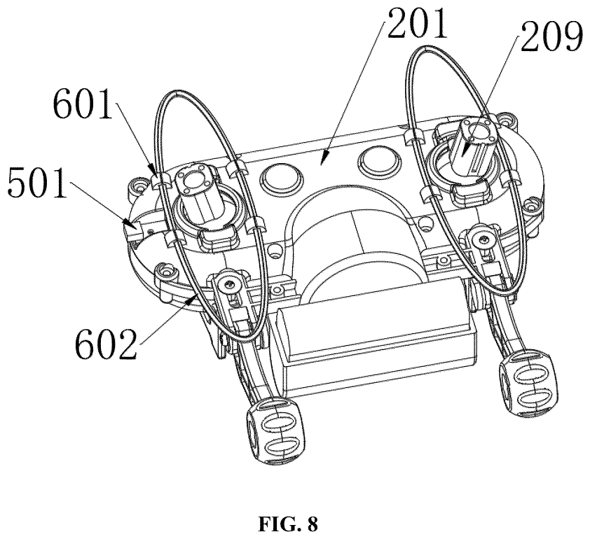

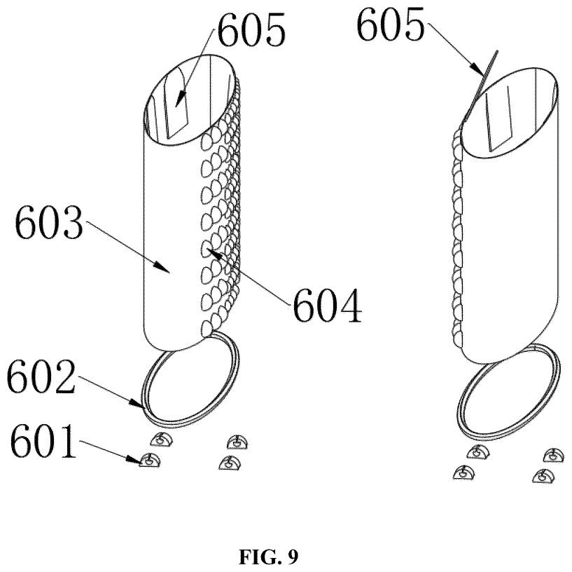

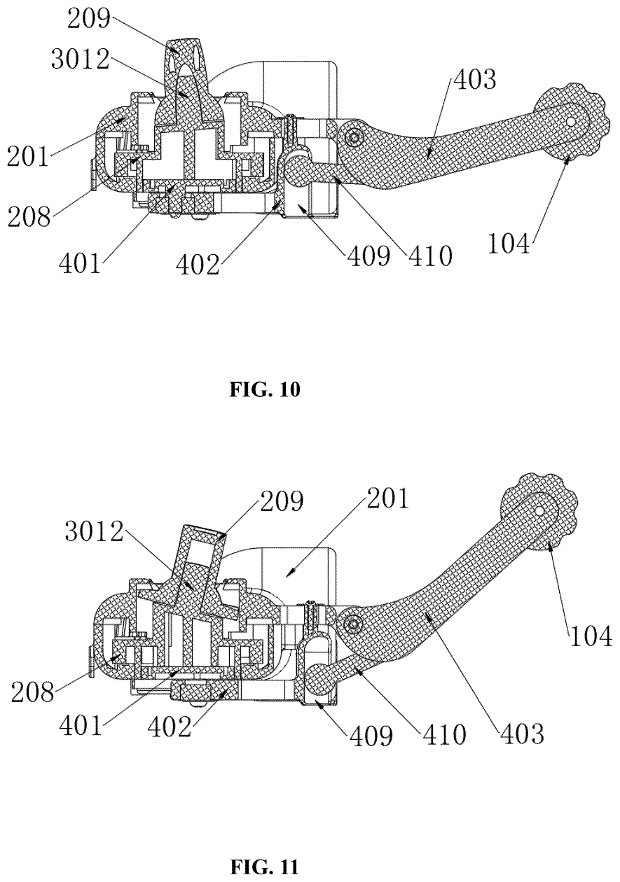

As shown in FIGS. 1-11, the present disclosure provides a massage device with swing arms of a portable sleep pillow. The massage device comprises a base 101, two neck massage heads 102, two shoulder massage heads 103, two trapezius massage heads 104, and a transmission portion.

The two neck massage heads 102 are obliquely disposed, and an included angle between the two neck massage heads 102 faces upward. The two shoulder massage heads 103 and the two trapezius massage heads 104 are horizontally disposed. The two shoulder massage heads 103 are disposed outside the two neck massage heads 102. The two trapezius massage heads 104 are respectively disposed below the two shoulder massage heads 103.

The transmission portion is disposed on the base 101 and configured to drive the two neck massage heads 102, the two shoulder massage heads 103, and the two trapezius massage heads 104 to perform kneading actions. Reversing pieces 301 are disposed inside the transmission portion. The reversing pieces 301 are configured to adjust the kneading actions of the two neck massage heads 102 and the two shoulder massage heads 103 to increase diversity of the kneading actions.

When the kneading actions are not performed, the two neck massage heads 102 are configured to support a neck of a user, and the two shoulder massage heads 103 and the two trapezius massage heads 104 do not contact the neck and shoulders of the user, so as to reduce discomfort of the user.

In the embodiment of the present disclosure, before using the massage device, the base 101 needs to be fixedly connected to a recess defined in an upper surface of a sponge pillow body 801, so as to facilitate mounting of the two neck massage heads 102, the two shoulder massage heads 103, and the two trapezius massage heads 104. In the off state, positions of the two neck massage heads 102 are adjusted to lowest positions relative to the base 101. When the user rests on the sponge pillow body 801, the two neck massage heads 102 support the neck, and the two shoulder massage heads 103 and the two trapezius massage heads 104 are located below the neck and do not contact the neck, so that the sponge pillow body 801 and the massage device as a whole fit and support the neck of the user. Therefore, when the user sleeps on the sponge pillow body 801 and the massage device for a long time, there is no discomfort caused by compression of the two shoulder massage heads 103 and the two trapezius massage heads 104.

When kneading massage is needed, the neck, the shoulders and the trapezius muscles are massaged by the two neck massage heads 102, the two shoulder massage heads 103, and the two trapezius massage heads 104.

Through cooperation of the transmission portion and the two reversing pieces 301, the massage technique of the massage device is diversified.

Furthermore, a protective shell 201 is fixedly connected to an upper surface of the base 101. Two first swing arms 210 are disposed on the protective shell 201. The two neck massage heads 102 are respectively fixedly connected to surfaces of the two first swing arms 210. The two shoulder massage heads 103 are respectively disposed on end portions of the two first swing arms 210. universal ball racks 209 are respectively fixedly disposed on bottom portions of the two first swing arms 210. Ball grooves 211 are defined in a surface of the protective shell 201. Limiting linear grooves 212 are symmetrically defined in an inner wall surface of each of the ball grooves 211. Limiting protruding blocks 213 are symmetrically and fixedly connected to a surface of each of the universal ball racks 209. A spherical part of each of the universal ball racks 209 is disposed in a corresponding one of the ball grooves 211. The limiting protruding blocks 213 are respectively disposed in the limiting linear grooves 212. The reversing pieces 301 are disposed below the two universal ball racks 209.

A battery compartment 202 is mounted on the surface of the protective shell 201. A brushless motor 203 is mounted inside the protective shell 201. A spiral gear rod 204 is fixedly connected to an end portion of an output shaft of the brushless motor 203. First rotating shafts 205 are symmetrically disposed inside the protective shell 201. The first rotating shafts 205 are rotatably connected to the protective shell 201 through circular grooves 214 symmetrically defined in an inner wall of the protective shell 201. The massage device further comprises two helical gears 206 and two . first gears 207 A surface of each of the first rotating shafts 205 is fixedly connected to a corresponding one of the helical gears 206 and a corresponding one of the first gears 207 from top to bottom. The two helical gears 206 are engaged with the spiral gear rod 204. Two second gears 208 are symmetrically disposed inside the protective shell 201. Each of the second gears 208 is engaged with a corresponding one of the first gears 207, and the reversing pieces 301 are respectively disposed on surfaces of the two second gears 208.

Each of the second gears 208 is rotatably disposed in a corresponding one of the circular grooves 214 through a protruding ring disposed on a bottom surface thereof.

The reversing pieces 301 comprise a first reversing block 3011 and a second reversing block 3012. The first reversing block 3011 and the second reversing block 3012 are respectively disposed on the surfaces of the two second gears 208. First cylinders are respectively protruded from an inclined surface of the first reversing block 3011 and an inclined surface of the second reversing block 3012. Each of the first cylinders is inserted into a first columnar groove defined in a bottom surface of a corresponding one of the universal ball racks 209.

The first reversing block 3011 is inserted into a second columnar groove defined in an upper surface of a first one of the two second gears 208 through a second cylinder protruding from a bottom surface of the first reversing block 3011. The second reversing block 3012 is fixedly connected to an upper surface of a second one of the two second gears 208. An adjusting groove 3013 is defined in the first reversing block 3011. The adjusting groove 3013 is in an incomplete ring shape. An adjusting block 3014 is disposed in the adjusting groove 3013. The adjusting block 3014 is fixedly connected to the first one of the two second gears 208.

In the embodiment, each of the universal ball racks 209 is positioned in the corresponding one of the ball grooves 211 by the two limiting protruding blocks 213 thereof and the corresponding limiting linear grooves 212. The inclined surface of the first reversing block 3011 compresses a bottom surface of a first universal ball rack 209, and the inclined surface of the second reversing block 3012 compresses a bottom surface of a second universal ball rack 209, so that each of the universal ball racks 209 make multi-angle movements inside the corresponding one of the ball grooves 211. The two reversing pieces 301 respectively adjust the kneading actions of the two neck massage heads 102, the two shoulder massage heads 103 and the two trapezius massage heads 104. Specifically, when the brushless motor 203 rotates, the spiral gear rod 204 rotates in one direction, and the spiral gear rod 204 rotates to drive the two first gears 207, the two helical gears 206, and the two first rotating shafts 205 to rotate, thereby driving the two second gears 208 to rotate.

The rotation of the second one of the second gears 208 drives the second reversing block 3012 to rotate accordingly, thereby driving a second one of the first swing arms 210 to swing through the first cylinder on the inclined surface of the second reversing block 3012 and the second universal ball rack 209. The first one of the second gears 208 in contact with the first reversing block 3011 abuts against a first side wall of the adjusting groove 3013 through the adjusting block 3014, thereby driving the first reversing block 3011 to rotate synchronously, and then drives a first one of the first swing arms 210 to swing.

When the brushless motor 203 runs to drive the Spiral gear rod 204 to rotate in an opposite direction, the first one of the second gears 208 in contact with the first reversing block 3011 drives the adjusting block 3014 to rotate along a curved inner wall of the adjusting groove 3013 until the adjusting block 3014 abuts against a second side wall of the adjusting groove 3013. Then, the first reversing block 3011 and the first one of the second gears 208 rotate synchronously, thereby driving the first one of the first swing arms 210 to swing.

When the adjusting block 3014 abuts against the first side wall of the adjusting groove 3013, the first reversing block 3011 and the second reversing block 3012 are symmetrically disposed ins the protective shell 201, and the rotation of the spiral gear rod 204 causes the adjusting block 3014 to continue to abut against the first side wall of the adjusting groove 3013. The two first swing arms 210 move synchronously.

When the rotation of the spiral gear rod 204 causes the adjusting block 3014 to move toward the second side wall of the adjusting groove 3013, the movements of the two first swing arms 210 are not synchronized, and the two first swing arms 210 move asynchronously, that is, perform the asynchronous movements.

During the swinging of the two first swing arms 210, since the two shoulder massage heads 103 are disposed on the outside of the two neck massage heads 102, the shoulder massage heads 103 do not interfere with the neck when massaging the shoulders, so that when the user does not need the kneading massage, the shoulder massage heads 103 are well organized.

The massage device further comprises eccentric blocks 401, eccentric brackets 402, and second swing arms 403. Each of the eccentric blocks 401 is fixedly connected in the protruding ring on the bottom surface of a corresponding one of the second gears 208. Each of the eccentric brackets 402 is rotatably connected to a shaft disposed on a bottom surface of a corresponding one of the eccentric blocks 401 through a first bearing. Hinge seats 405 are symmetrically and fixedly connected to the surface of the protective shell 201. A U-shaped portion is disposed on a surface, away from the protective shell 201, of each of the hinge seats 405, and each of the second swing arms 403 is hinged into a corresponding U-shaped portion. Sliding grooves 405 are defined in surfaces of the hinge seats 405. Sliding blocks 408 are respectively slidable in the sliding grooves 405. A bottom end of each of the sliding blocks 408 is fixedly connected to the surface of a corresponding one of the eccentric bracket. Guide ball head rods 410 are respectively connected to surfaces of the second swing arms 403. Guide grooves 409 are respectively defined in the eccentric brackets 402. A ball head of each of the guide ball head rods 410 is slidable in a corresponding one of the guide grooves 409. Each of the trapezius massage heads 104 is disposed on one end of a corresponding one of the second swing arms 403.

In the embodiment of the present disclosure, when the second gears 208 rotate, the eccentric blocks 401 are driven to rotate synchronously. The shaft portion of each of the eccentric blocks 401 is eccentrically disposed, so the eccentric blocks 401 respectively drives the eccentric brackets 402 to move. Each of the eccentric brackets 402 is fixedly connected to the corresponding one of the sliding blocks 408, and each of the sliding blocks 408 is limited by a corresponding one of the sliding grooves 406, so each of the eccentric brackets 402 performs linear reciprocating motion along the corresponding one of the sliding grooves 406 when the corresponding one of the eccentric blocks 401 rotates. The second swing arms 403 are hinged to the protective shell 201 through the hinge seats 405. During the reciprocating motion of each of the eccentric brackets 402, each of the guide grooves 409 compresses the corresponding one of the guide ball head rods 410 to make each of the second swing arms 403 rotate around a corresponding one of the hinge seats 405, thereby massaging the trapezius muscles of the user through the two trapezius massage heads 104.

When the user does not need massage, each of the eccentric brackets 402 is moved to a position farthest from the corresponding one of the hinge seats 405, so that each of the eccentric brackets 402 moves the corresponding one of the second swing arms 403 to a horizontal state through the corresponding one of the guide grooves 409 and the corresponding one of the guide ball head rod s410. At this time, the two second swing arms 403 and the two trapezius massage heads 104 are stored below the neck of the user and do not contact the neck, so that when the user needs to sleep on the massage device for a long time, the two trapezius massage heads 104 are not felt by the user.

A Hall sensor 501 is mounted on the surface of the protective shell 201. An induction magnetic column 502 is embedded in an upper surface of one of the second gears 208 closer to the Hall sensor 501. A printed circuit board (PCB) control box 503 is fixedly connected to the surface of the protective shell 201. A controller 504 is disposed on one side of the protective shell 201, and a heating lamp board 505 is disposed above the protective shell 201.

In the embodiment of the present disclosure, the positions of the neck massage heads 102, the shoulder massage heads 103 and the trapezius massage heads 104 are identified and recorded by the PCB control box 503 through the induction of the induction magnetic column 502 by the Hall sensor 501, and the controller 504 is configured to control an action of the massage device. The heating lamp board 505 is mounted above the protective shell 201. Specifically, the sponge pillow body 801 covers a surface of the protective shell 201 in an area that does not interfere with the two first swing arms 210, the two neck massage heads 102, the two shoulder massage heads 103, the two trapezius massage heads 104, the two eccentric brackets 402, and the two second swing arms 403. The heating lamp board 505 is fixed on the surface of the sponge pillow body block 801 at a position close to the neck of the user, and the heating lamp board 50 is configured to heat the neck, the shoulders, and trapezius muscles of the user, thereby providing a hot compress to the user. A heat insulation layer is coated on the heating lamp board 505 to protect the user's skin from high temperature.

It should be noted that the arrangements of the brushless motor 203, the Hall sensor 501, the induction magnetic column 502, the PCB control box 503, the controller 504, and the heating lamp board 505 requires power supply from the battery compartment 202. A circuit connection method, a working principle and a usage process of the battery compartment 202, the brushless motor 203, the Hall sensor 501, the induction magnetic column 502, the PCB control box 503, the controller 504, and the heating lamp board 505 are common knowledge in the prior art, which is not described in detail herein.

Cloth sleeves 603 are respectively disposed above the ball grooves 211. The cloth sleeves 603 are elastic to be elastically deformed under the kneading actions of the two first swing arms 210, each of the cloth sleeves 603 is configured to accommodate a corresponding one of the first swing arms 210, a corresponding one of the neck massage heads 102, and a corresponding one of the shoulder massage heads 103. Massage protrusion 604s are evenly and fixedly connected to outer surfaces of the cloth sleeves 603. Sizes of the massage protrusion 604s gradually increase along a circumference of each of the cloth sleeves 603.

Furthermore, limiting buckles 601 are symmetrically and fixedly disposed on two sides of each of the ball grooves 211 defined on the surface of the protective shell 201. Elastic supporting strips 602 are disposed above the ball grooves 211, each of the elastic supporting strips 602 is slidably connected to corresponding limiting buckles 601 through T-shaped limiting grooves respectively defined in surfaces of the corresponding limiting buckles 601. Each of the cloth sleeves 603 is fixedly connected to an upper surface of a corresponding one of the elastic supporting strips 602.

When the two first swing arms 210 drive the two neck massage heads 102 to perform the kneading actions, the massage protrusion 604s massage the neck. By adjusting positions of the elastic supporting strips 602 relative to the limiting buckles 601, the massage protrusion 604s do not contact the neck of the user or at least partially contact the neck of the user.

Fixed cloth strips 605 are evenly and fixedly connected to an inner wall of each of the cloth sleeves 603. The fixed cloth strips 605 of each of the cloth sleeves 603 are disposed on one end of each of the cloth sleeves 603 away from the corresponding one of the elastic supporting strips 602. The fixed cloth strips 605 of each of the cloth sleeves 603 are configured to fix the one end of each of the cloth sleeves 603 to one side of the massage device away from the protective shell 201.

In the embodiment, when the user sleeps on the massage device for a long time, the cloth sleeves 603 prevent the neck skin of the user from contacting the two first swing arms 210, the two shoulder massage heads 103, and the two neck massage heads 102, prevent sweat and dust from remaining on surfaces of the two first swing arms 210, the two neck massage heads 102 and the two shoulder massage heads 103, and also prevent the dust from entering the ball grooves 211. In addition, the user is able to move each of the elastic supporting strips 602 clamped in the corresponding limiting buckles 601 according to his/her own needs, so that the massage protrusions 604 on each of the cloth sleeves 603 contact the neck skin. During a swinging process of the two first swing arms 210, the massage protrusions 604 performs auxiliary massage on the neck. When the auxiliary massage of the massage protrusions 604 is not needed, each of the cloth sleeves 603 is rotated to expose an area where the massage protrusions 604 are not provided, so that an exposed cloth surface of each of the cloth sleeves 603 directly fits the neck skin of the user.

When the cloth sleeves 603 needs to be replaced or cleaned, a force is applied to each of the cloth sleeves 603 to move away from a corresponding one of the elastic supporting strips 602, so that each of the elastic supporting strips 602 is elastically deformed and separates from the corresponding limiting buckle 601. When each of the cloth sleeves 603 needs to be installed, each of the elastic supporting strips 602 is compressed to elastically deform and be pressed into corresponding T-shaped limiting groove s of the corresponding limiting buckle 601.

A hook surface of a Velcro is fixed on one side of each of the fixed cloth strips 605 facing the inner wall of the corresponding one of the cloth sleeves 603, and a fur surface of the Velcro is fixed on the upper surface of the sponge pillow body 801. By pressing one of the fixed cloth strips 605 with the fur surface in each of the cloth sleeves 603, one end of each of the cloth sleeves 603 is fixed on the upper surface of the sponge pillow body 801. Therefore, the massage protrusions 604 on the outer surface of each of the cloth sleeves 603 or the cloth surface of each of the cloth sleeves 603 well fit the neck skin of the user.

It should be noted that a spherical surface of each of the universal ball racks 209 is specifically hemispherical, and the bottom surface thereof is a circular flat surface. A top of each hemisphere is a columnar protrusion, which facilitates each of the universal ball racks 209 to rotate at different angles inside the corresponding one of the ball grooves 211.

Each of the shoulder massage heads 103 is fixedly connected to the corresponding one of the first swing arms 210 or each of the shoulder massage heads 103 is rotatably connected to the corresponding one of the first swing arms 210 through a corresponding second bearing. Each of the trapezius massage heads 104 is fixedly connected to the corresponding one of the second swing arms 403, or each of the trapezius massage heads 104 is rotatably connected to the corresponding one of the second swing arms 403 through a corresponding third bearing. The user is able to select a specific connection method according to needs.

The above are optional embodiments of the present disclosure. It should be pointed out that for those skilled in the art, improvements and modifications can be made without departing from the principles of the present disclosure. These improvements and modifications should also fall within the protection scope of the present disclosure.

What is claimed is:

1. A massage device with swing arms of a portable sleep pillow, comprising:
a base;
two neck massage heads;
two shoulder massage heads;
two trapezius massage heads; and
a transmission portion;
wherein the two neck massage heads are obliquely disposed, and an included angle between the two neck massage heads faces upward, the two shoulder massage heads and the two trapezius massage heads are horizontally disposed, the two shoulder massage heads are disposed outside the two neck massage heads, and the two trapezius massage heads are respectively disposed below the two shoulder massage heads;
wherein the transmission portion is disposed on the base and configured to drive the two neck massage heads, the two shoulder massage heads, and the two trapezius massage heads to perform kneading actions, reversing pieces are disposed inside the transmission portion, and the reversing pieces are configured to adjust the kneading actions of the two neck massage heads and the two shoulder massage heads;
wherein when the kneading actions are not performed, the two neck massage heads are configured to support a neck of a user, and the two shoulder massage heads and the two trapezius massage heads do not contact the neck and shoulders of the user.

2. The massage device according to claim 1, wherein a protective shell is fixedly connected to an upper surface of the base, two first swing arms are disposed on the protective shell, the two neck massage heads are respectively fixedly connected to surfaces of the two first swing arms, the two shoulder massage heads are respectively disposed on end portions of the two first swing arms, universal ball racks are respectively fixedly disposed on bottom portions of the two first swing arms, ball grooves are defined in a surface of the protective shell, limiting linear grooves are symmetrically defined in an inner wall surface of each of the ball grooves, limiting protruding blocks are symmetrically and fixedly connected to a surface of each of the universal ball racks, a spherical part of each of the universal ball racks is disposed in a corresponding one of the ball grooves, the limiting protruding blocks are respectively disposed in the limiting linear grooves, and the reversing pieces are disposed below the two universal ball racks.

3. The massage device according to claim 2, wherein a battery compartment is mounted on the surface of the protective shell, a brushless motor is mounted inside the protective shell, a spiral gear rod is fixedly connected to an end portion of an output shaft of the brushless motor, first rotating shafts are symmetrically disposed inside the protective shell, and the first rotating shafts are rotatably connected to the protective shell through circular grooves symmetrically defined in an inner wall of the protective shell,
wherein the massage device further comprises two helical gears and two first gears, a surface of each of the first rotating shafts is fixedly connected to a corresponding one of the helical gears and a corresponding one of the first gears from top to bottom, the two helical gears are engaged with the spiral gear rod, two second gears are symmetrically disposed inside the protective shell, each of the second gears is engaged with a corresponding one of the first gears, and the reversing pieces are respectively disposed on surfaces of the two second gears.

4. The massage device according to claim 3, wherein each of the second gears is rotatably disposed in a corresponding one of the circular grooves through a protruding ring disposed on a bottom surface of the corresponding one of the circular grooves;
wherein the reversing pieces comprise a first reversing block and a second reversing block, the first reversing block and the second reversing block are respectively disposed on the surfaces of the two second gears, first cylinders are respectively protruded from an inclined surface of the first reversing block and an inclined surface of the second reversing block, and each of the first cylinders is inserted into a first columnar groove defined in a bottom surface of a corresponding one of the universal ball racks.

5. The massage device according to claim 4, wherein the first reversing block is inserted into a second columnar groove defined in an upper surface of a first one of the two second gears through a second cylinder protruding from a bottom surface of the first reversing block, the second reversing block is fixedly connected to an upper surface of a second one of the two second gears, an adjusting groove is defined in the first reversing block, the adjusting groove is in an incomplete ring shape, an adjusting block is disposed in the adjusting groove, and the adjusting block is fixedly connected to the first one of the two second gears.

6. The massage device according to claim 5, wherein the massage device further comprises eccentric blocks, eccentric brackets, and second swing arms, each of the eccentric blocks is fixedly connected in the protruding ring on the bottom surface of a corresponding one of the second gears, each of the eccentric brackets is rotatably connected to a shaft disposed on a bottom surface of a corresponding one of the eccentric blocks through a first bearing, and hinge seats are symmetrically and fixedly connected to the surface of the protective shell;
wherein a U-shaped portion is disposed on a surface, away from the protective shell, of each of the hinge seats, and each of the second swing arms is hinged into a corresponding U-shaped portion;
wherein sliding grooves are defined in surfaces of the hinge seats, sliding blocks are respectively slidable in the sliding grooves, a bottom end of each of the sliding blocks is fixedly connected to the surface of a corresponding one of the eccentric brackets, guide ball head rods are respectively connected to surfaces of the second swing arms, guide grooves are respectively defined in the eccentric brackets, a ball head of each of the guide ball head rods is slidable in a corresponding one of the guide grooves, and each of the trapezius massage heads is disposed on one end of a corresponding one of the second swing arms.

7. The massage device according to claim 6, wherein a Hall sensor is mounted on the surface of the protective shell, an induction magnetic column is embedded in an upper surface of one of the second gears closer to the Hall sensor, a printed circuit board (PCB) control box is fixedly connected to the surface of the protective shell, a controller is disposed on one side of the protective shell, and a heating lamp board is disposed above the protective shell.

8. The massage device according to claim 2, wherein cloth sleeves are respectively disposed above the ball grooves, the cloth sleeves are elastic to be elastically deformed under the kneading actions of the two first swing arms, each of the cloth sleeves is configured to accommodate a corresponding one of the first swing arms, a corresponding one of the neck massage heads, and a corresponding one of the shoulder massage heads, massage protrusions are evenly and fixedly connected to outer surfaces of the cloth sleeves, and sizes of the massage protrusions gradually increase along a circumference of each of the cloth sleeves.

9. The massage device according to claim 8, wherein limiting buckles are symmetrically and fixedly disposed on two sides of each of the ball grooves defined on the surface of the protective shell, elastic supporting strips are disposed above the ball grooves, each of the elastic supporting strips is slidably connected to corresponding limiting buckles through T-shaped limiting grooves respectively defined in surfaces of the corresponding limiting buckles, and each of the cloth sleeves is fixedly connected to an upper surface of a corresponding one of the elastic supporting strips;
wherein when the two first swing arms drive the two neck massage heads to perform the kneading actions, the massage protrusions massage the neck, and by adjusting positions of the elastic supporting strips relative to the limiting buckles, the massage protrusions do not contact the neck of the user or at least partially contact the neck of the user.

10. The massage device according to claim 9, wherein fixed cloth strips are evenly and fixedly connected to an inner wall of each of the cloth sleeves, the fixed cloth strips of each of the cloth sleeves are disposed on one end of each of the cloth sleeves away from the corresponding one of the elastic supporting strips, and the fixed cloth strips of each of the cloth sleeves are configured to fix the one end of each of the cloth sleeves to one side of the massage device away from the protective shell.
